(19) United States
(12) Patent Application Publication
(10) Pub. No.: US 2011/0251572 A1
(43) Pub. Date: Oct. 13, 2011

(54) Urine Collection Device

(75) Inventors: Vasu Nishtala, Snellville, GA (US); Jean Chery, Stockbridge, GA (US); David Fish, Covington, GA (US)

(73) Assignee: C.R. Bard, Inc., Murray Hill, NJ (US)

(21) Appl. No.: 12/996,595

(22) PCT Filed: Jun. 5, 2009
(86) PCT No.: PCT/US09/46472

§ 371 Date: Jun. 29, 2011

Related U.S. Application Data
(60) Provisional application No. 61/059,629, filed on Jun. 6, 2008.

(51) Int. Cl. A61F 5/44

(52) U.S. Cl. 604/323; 604/326; 604/324

(57) ABSTRACT

A fluid collection assembly including a container having an interior chamber for collecting fluid, and a quick connect device coupled to the top of the container to couple to a mated connection device on a drainage tube. The quick connect device and the mated connection device may be in an open configuration to permit fluid flow when engaged and in a closed configuration to prevent fluid flow when disengaged. An adjustable hanger may be attached to the container, including an adjustable member that vertically repositions the container.

### PRIORITY

This application claims the benefit of priority to U.S. Provisional Patent Application No. 61/059,629, filed Jun. 6, 2008, which is incorporated by reference into this application as if fully set forth herein.

### BACKGROUND

Hospitals currently use urinary drainage bags for collecting urine from catheterized patients. These bags permit the patient to remain in bed, without having to be moved to use a bathroom or a bedpan. Once the bag is full, a drainage system may be used to empty the bag. Generally, the tubing connected to the catheter is disconnected close to the insertion point in the patient. A new bag is then connected, reconnecting the tubing to the same catheter insertion point. Disconnecting and reconnecting open tubes near a patient may contaminate the connection locations, resulting in potential infection. Simply disconnecting and reconnecting open tubes holding fluid may become messy as the tubes may drip during the disconnect and reconnect process. Changing the connections close to the patient may increase potential exposure to the patient and result in infection.

Typically, the holding bags for the urine are attached to the patient's bed, generally, below the level of the patient so that the urine flows into the bag under the force of gravity. The bag may hang down from a hook or other attachment at the end of the patient's bed. However, there has been a recognized advantage to lowering a patient's bed. Therefore, as the bed is lowered, the urine bag gets closer to the ground. Having medical devices attached to a patient touching the ground may not be desired. In addition, the hoses connecting the patient to the bag may also drag along the ground, increasing potential infection and causing potential hazards to nurses and doctors walking near a patient.

### BRIEF SUMMARY

Described herein is a fluid collection assembly. The fluid collection assembly may include a container having an interior chamber for collecting fluid; and a quick connect device coupled to the top of the container. The quick connect can be designed to couple to a mated connection device on a drainage tube. In some embodiments, the quick connect device and the mated connection device are in an open configuration to permit fluid flow when engaged and are in a closed configuration to prevent fluid flow when disengaged.

In one embodiment, the container includes an outlet device coupled to the bottom of the container, the outlet device being in a normally closed configuration. The outlet device may occlude an outlet device tube. For example, the outlet device can include a valve, such as, for example, a trumpet valve, a duckbill valve or a pushbutton valve.

In one embodiment, the outlet device includes an interface to couple to a disposal container. In some example systems, the outlet device must be connected to a fluid disposal container in order for fluid to flow through the outlet device and drain from the container. The container can include a restraining device or retaining device, such as a housing to retain the outlet device.

In one embodiment, a quick connect device includes a male/female connection pair. An open pathway can be provided when the quick connect device is engaged. An element may seal the pathway when the quick connect device is disengaged. In one example, the quick connect device may include a duckbill valve and a female receiving portion to receive the duckbill valve and form a connection. The quick connect device may also include a vented area. The vent may minimize urine stasis in the drain tube.

In one embodiment, a fluid collection bag and drain assembly includes a container having an interior chamber for collecting fluid. The assembly may also include an outlet device coupled to the bottom of the container wherein the outlet device is in a normally closed configuration. The outlet may also include a retaining device to retain the outlet device.

In one embodiment, an adjustable hanger attached to a fluid collection bag includes an adjustable member that repositions the fluid collection bag with respect to a surface underneath the fluid collection bag (i.e., vertically repositions the collection bag). The adjustable hanger can include a hook section to couple the hanger to a bed, and a bag connection device to hold the fluid collection bag. The bag connection device may comprise a hook or a slip hole. In one example system, the adjustable member may comprise an arm. The arm may include a hinge. In another example, the arm may be a telescoping arm. Hinges and telescoping arms can allow the hanger to be adjusted.

### BRIEF DESCRIPTION OF THE DRAWINGS

The multiple drawings refer to embodiments of the invention. While embodiments of the invention described herein are subject to various modifications and alternative forms, specific embodiments thereof have been shown by way of example in the drawings and will herein be described in detail.

FIG. 1 illustrates a representative urine collection system according to embodiments described herein.

FIG. 2 illustrates a representative quick connect assembly according to embodiments described herein.

FIG. 3 illustrates a representative outlet device according to embodiments described herein.

FIGS. 4A-4E illustrate representative adjustable hangers to support the urine collection bag from a patient's bed according to embodiments described herein.

### DETAILED DESCRIPTION

The following detailed description should be read with reference to the drawings. The drawings, which are not necessarily to scale, depict selective embodiments and are not intended to limit the scope of the invention. The detailed description illustrates by way of example, not by way of limitation, the principles of the invention. This description describes several embodiments, adaptations, variations, alternatives, and uses of the invention, including what is presently believed to be the best mode of carrying out the invention.

Embodiments described herein relate generally to urinary collection devices. The urinary collection bag may be an expandable bag connected to a drain tube and then to a catheter. A fluid communication path is created between the collection bag and a patient's bladder. A quick connect feature may be used between the bag and the drain tubing to close the connection ends during the disconnect and reconnect transition. The quick connect may include a vented area on the device, minimizing urine stasis in the drainage tube. This may permit exchanging bags without leaking or dripping. Additionally, distancing the connection location from the patient may minimize risk of infection. A push button outlet device may additionally or alternatively be included to assist with emptying a collection bag. The outlet device may integrate with a disposal canister to prevent contact with urine during disposal. The outlet device may incorporate a push button to prevent the flow of urine during disposal until desired by a user. Another aspect, used in combination with other aspects described herein or independently thereof, is an adjustable length hanger for raising or lowering the urine bag. For instance, when a bed is lowered, the adjustable length hanger permits the bag to be adjusted to prevent or reduce contact with the floor.

As described herein, any of the combinations of these features (e.g., quick connect, outlet device connection, the adjustable hanger, or the like), may be used in any combination or sub-combination to form a urinary collection device. Further, any of these components may be part of a urinary collection system and may be used to collect urine from a subject in need thereof. Unless otherwise indicated, the embodiments described herein need not be limited to applications in humans. As one of skill in the art would appreciate, variations of embodiments described herein may be applied to other mammals as well. Moreover, it should be understood that embodiments described herein may be applied in combination with any appropriate catheter, including but not limited to Foley catheters. Urinary catheters may include any tube or tube-like structure that provides access to the bladder. Urinary collection systems and devices, as described herein, may be used to collect urine from any subject in need thereof. A subject may include any appropriate user, including a medical patient. It must also be noted that, as used in this description, the singular forms “a,” “an,” and “the” include plural referents unless the context clearly dictates otherwise. Thus, for example, the term “a catheter” is intended to mean a single catheter or a combination of catheters, “a fluid” is intended to mean one or more fluids, or a mixture thereof.

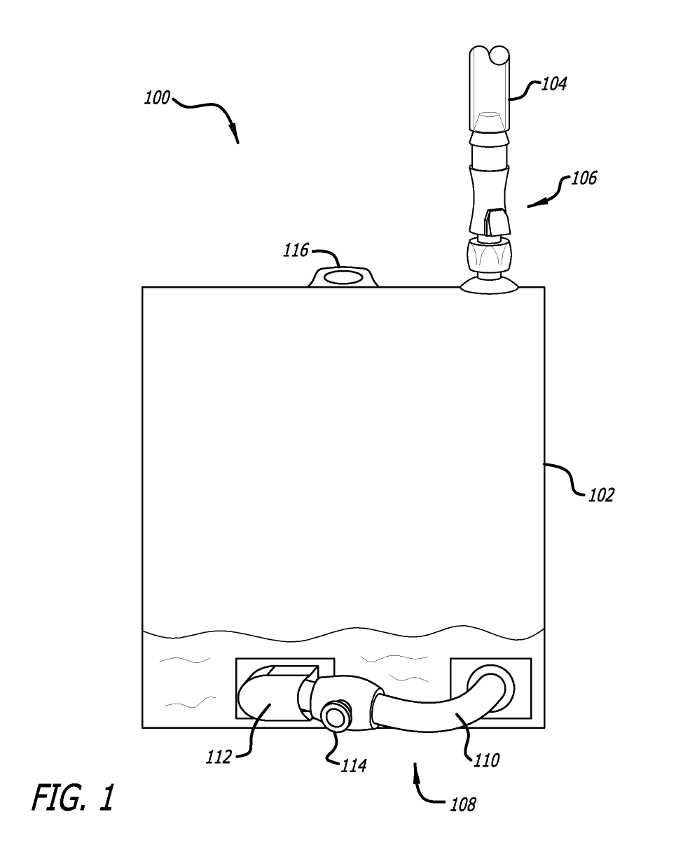

FIG. 1 illustrates a representative urine collection system 100 including a urine collection bag 102. The collection bag 102 may be attached to the patient through a drain tube 104, or tube-like system. The drain tube 104 may then be coupled to the catheter system leading to the patient's bladder. Therefore, a fluid connection path is created between the urine collection bag 102 and the patient's bladder. The bag 102 is then used as a receptacle to collect urine. The bag 102 may then be disconnected and replaced as it is filled. Alternately, or in conjunction with the disconnect, the bag 102 may be drained and used again.

The urine collection bag 102 may be of any conventional design. For instance, the bag 102 may include front and back sheets of a flexible, impermeable plastic such as polyvinyl chloride, heat welded around their peripheries to form a central collection chamber therebetween. Alternatively, the urine collection bag 102 may be an expandable container, which includes an open or expanded configuration, and a closed or collapsed configuration. The collection bag 102 may include a wide base region so that urine may be stored in the container 102 away from the inlet. The urine collection bag 102 may also be shaped or adapted to connect with a meter system, or any other appropriate device. The urine collection bag 102 may be of various shapes or volumes as well and is not limited to a rectangular shape.

A quick connect device 106 may be coupled to the collection bag 102. The quick connect 106 may assist in easily connecting and disconnecting the collection bag 102 from the drain tubing 104 leading to the patient. When the quick connect 106 is released, disconnecting the bag 102 from the tubing 104, the ends may close to prevent leakage. The interconnect system 106 may be used with urine bags 102 with meters to permit interchanging urine bags 102 with meters. Locating the detachment location 106 closer to the urine bag 102 removes the disconnect away from the patient. The risk of infection may be minimized by detaching the urine bag 102 at the distal end of the patient.

An outlet device 108 may additionally or alternatively be coupled to the collection bag 108. The outlet device 108 may integrate with a disposal canister, and therefore, reduce contact with urine during disposal. The outlet device 108 may include a tube 110 coupled to the urine collection bag 102. While the bag 102 is in use, the tube 110 of the outlet device 108 may be bent, and stored in a housing 112 coupled to the urine collection bag 102 to reduce the profile of the overall system 100. The outlet device 108 may also include a push button 114 or other mechanical device to prevent flow until the bag 102 is properly connected to the discharge container and ready to empty. The user may push the button 114 to open the flow path, once the outlet device 108 is properly connected to the disposal container.

An adjustable hanger may additionally or alternatively be used in conjunction with the collection bag 102. For example, the collection bag 102 may include a hole or tab 116 at the top to support the bag 102 from the hanger. An adjustable hanger may be achieved by using a sliding telescopic hanger mounted on a sleeve. The urine meter may then be raised when a bed is lowered to reduce the potential that the bag may contact the floor. Alternative embodiments of the design include a flip mechanism to raise the urine bag.

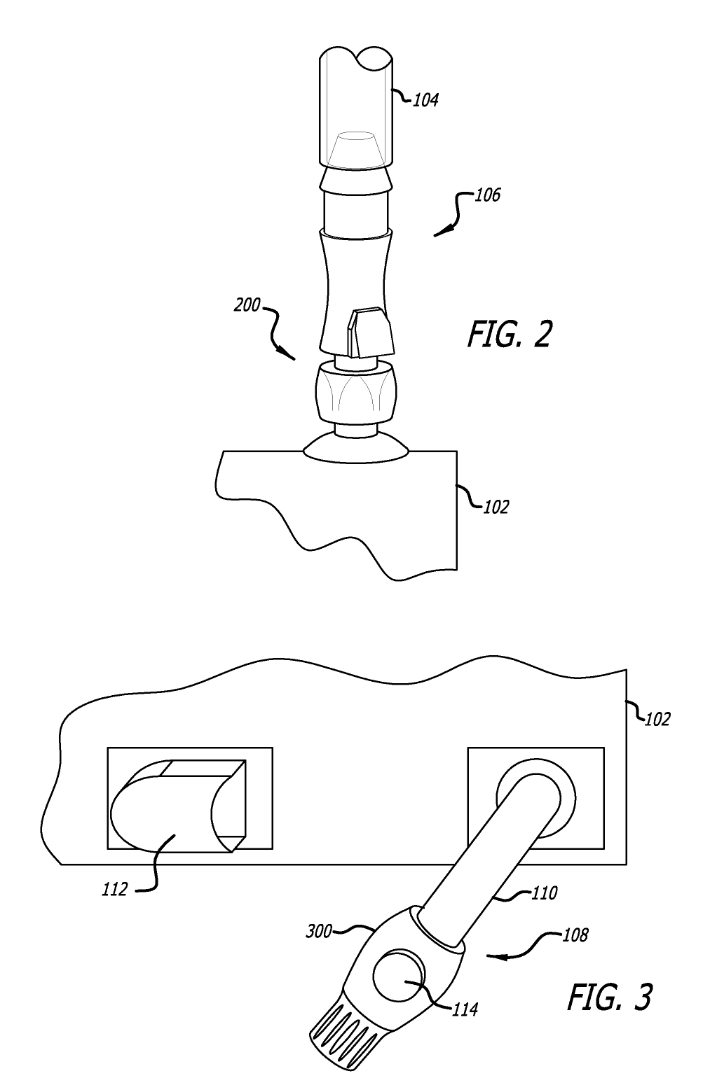

FIG. 2 illustrates a representative quick connect assembly 106 according to embodiments of the design. A male/female connection pair 200 may be coupled between the collection bag 102 and the drain tube 104 from the patient. The connection pair 200 permits the collection bag 102 to be attached and removed from the drain tube 104, quickly and easily. When the male/female portions 200 are engaged, an open pathway or conduit is provided for the urine to drain from the drain tube 104 to the reservoir of the collection bag 102. The male/female portion 200 may engage through a variety of actuations. For example, to engage the male/female connection portions 200, the two sections may rotationally twist, or may slide longitudinally along the axis of the connect portion relative to each other. Alternatively, there may be a combination of rotary twist which slides from the side. The quick connect 106 may additionally, or alternatively include a vented area on the device, minimizing urine stasis in the drainage tube 104.

In an alternate embodiment, when the male/female connection portions 200 are disengaged, an element seals the drain tube 104 to prevent leaking before the connection is fully broken. When engaged, the connection portion 200 may open the seal so urine may drain, unobstructed, into the attached urine collection bag 102. For example, a patient may be catheterized coupled to a collection bag 102 utilizing embodiments describe herein. When the bag 102 is full, the quick connect 106 is disengaged. An element seals off the drain tube 104 to prevent leaks when disconnected from the collection bag 102. The bag 102 may be emptied and reconnected, or a new bag connected. When connected to the new bag, the quick connect 106 is engaged, and the flow path is re-opened.

In one embodiment, the quick connect 106 may include a duckbill valve. The male portion may include a button that allows the connection or disconnection. Within the housing of the male portion, a duckbill may be included pointing toward the drain tube to shut off flow. The female portion may then be a plastic piece that goes around the duckbill of the male portion to prop open the duckbill and establish a flow path. Removing the female portion will then close the flow path.

Embodiments of the design permit modularity to change out urine bags 102 without disconnecting the drain tube 104 or interfering with the catheter/drain tube connection. When a person is generally admitted to an emergency room, the patient may be connected to a closed system including only a collection bag. Generally, a patient with a closed system has the catheter attached to a drain tube with a temporary seal at the juncture between the catheter and the drain tube, then the drain tube is attached to the collection system. If the person needs to be observed and is moved, the collection bag is changed for a meter bag to accurately monitor the patient's urine output. Traditionally, the urine bag and the drain tube would be removed from the patient near the catheter junction, breaking the catheter seal, and then reattaching a meter bag and drain tube at the same junction. The change has traditionally occurred upstream, near the patient. This may increase the risk of infection to a patient. Embodiments of the current design 100 may be used to move the disconnect location further downstream from the patient. The disconnect may occur between the drain tube 104 and the collection bag 102, which may be as far as the length of the drain tube 104 (e.g., approximately 5 feet). The quick connect 106, according to embodiments of the design, which attaches the tube 104 to the bag 102 at the disconnect may reduce spilling or leaking during the transfer, further preventing the chance of infection or contamination.

FIG. 3 illustrates a representative outlet device 108 according to embodiments of the design. The outlet device 108 may prevent flow through the outlet device 108 until it is connected to the disposal canister. The outlet device 108 may be connected to the bottom of the collection bag 102 and may occlude the tube 110 of the outlet device 108 until it is properly connected to the disposal canister 102. A valve 300 in the outlet device may be activated to open the tube and permit flow therethrough. In one embodiment, the end of the outlet device 108 may include a custom interface to couple with a disposal canister. The custom interface may enhance the seal between the outlet device and the disposal canister, minimizing leaks and enhancing the connection.

The outlet device 108 may be actively engaged during the drainage process to open the flow path. For example, a valve 300 within the outlet device 108 may require the user to actively engage the valve 300 during the entire drainage period. In one embodiment, the valve 300 may include a push button 114 that is normally closed. When the button 114 is engaged, the valve 300 is opened, and the urine may flow between the collection bag 102 and the disposal canister. When the button 114 is no longer engaged, the tube 110 is resealed, preventing further flow. The valve 300 may alternatively not require active engagement during the flow process, but may include two states. Once the outlet device 108 is connected, the valve 300 may be changed to permit flow between the collection bag 102 and the canister. When the collection bag 102 is empty, the state of the valve 300 is changed to re-seal the flow path.

In one embodiment, a push button 114 may be used to open the flow, once the collection bag 102 is connected to the disposal canister. The push button 114 permits one-handed use, and maintains a low-profile for the outlet device. A trumpet valve may be used to shut off the flow from the bag 102 to the outlet device. In an alternate embodiment, a duckbill valve may be used as described above. The duckbill valve may open the flow path when the outlet device is engaged with the disposal canister. When the disposal canister is disconnected from the outlet device, the flow path is occluded and minimizes spills or drips.

In one embodiment, the collection bag 102 may include a retaining device, to reduce the profile of the bag when in use with a patient. The retaining device may permit the end of the outlet device to rest within a holder 112. When the bag 102 is ready to be emptied, the outlet device tube 110 may be removed from the retaining device and connected to the disposal canister. The retaining device may be a housing in which the outlet device tube rests. Another embodiment may include a restraining device, such as a loop or other mechanical connection to bring the output device tube along the side of the collection bag.

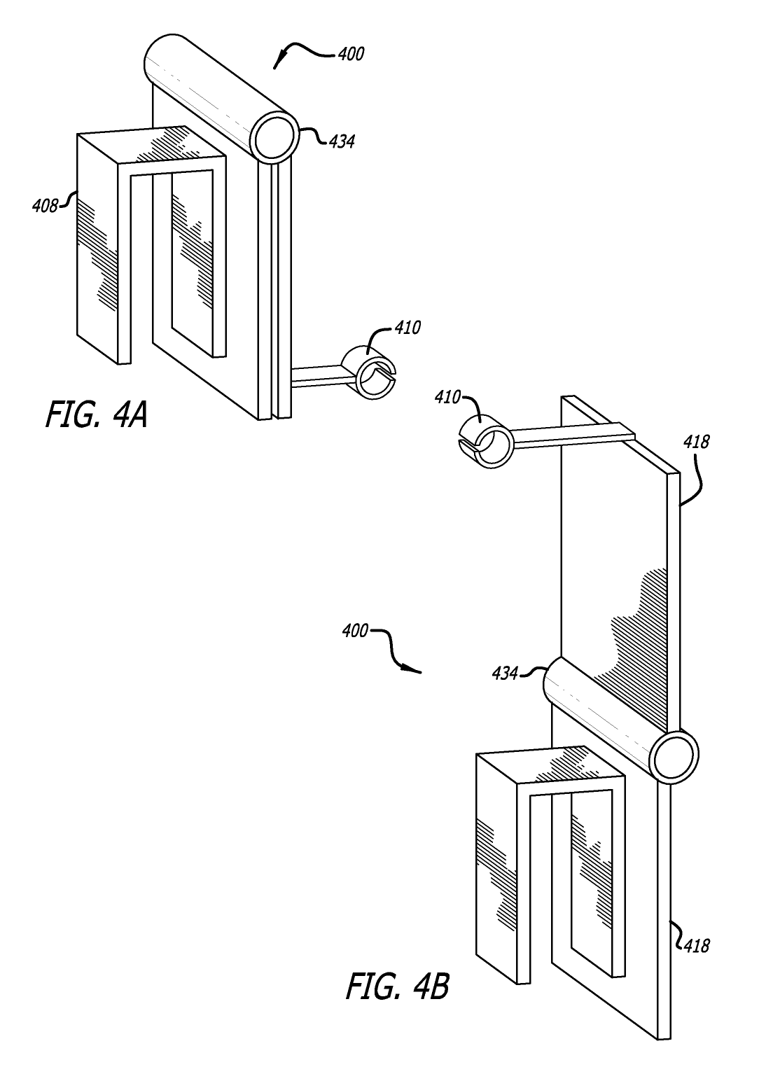

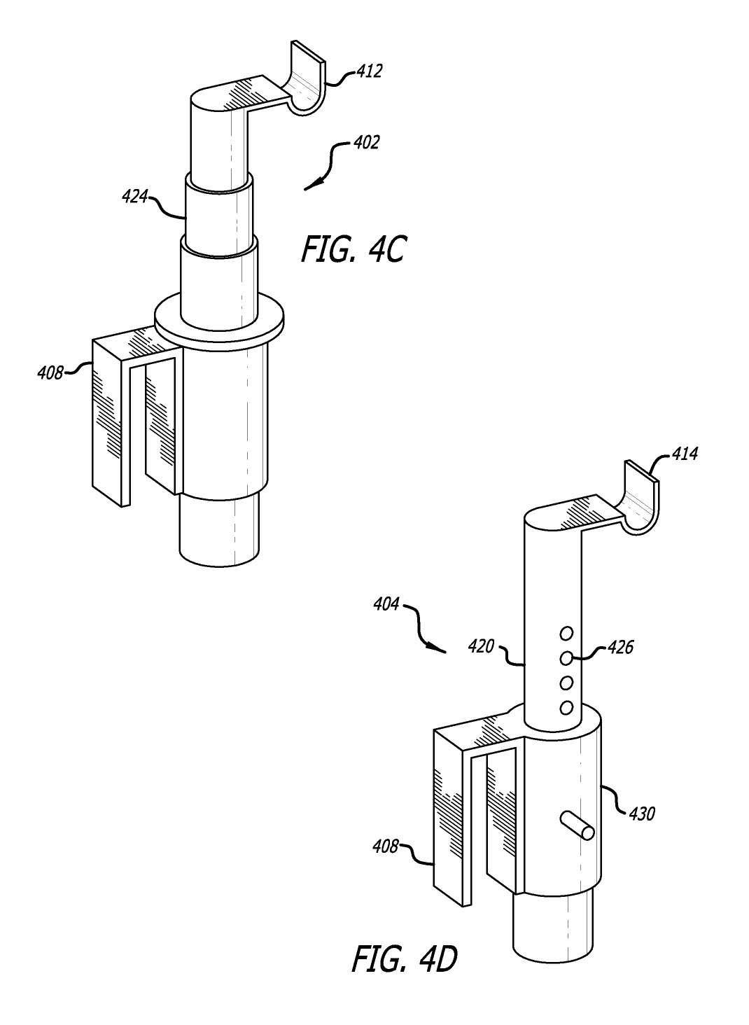

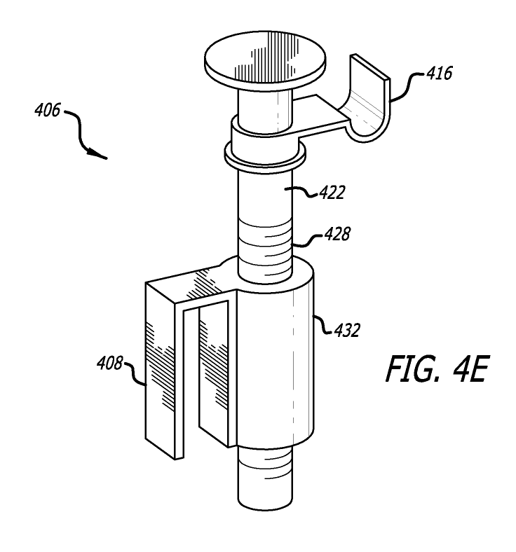

FIGS. 4A-4E illustrate representative adjustable hangers 400, 402, 404, 406 to support the urine collection bag from a patient's bed. The adjustable hanger may include a hook section 408 on one side to couple the hanger to a patient's bed. A different side of the hanger may include a bag connection device, such as, for example, a smaller or differently configured hook section, to hold the collection bag. The collection bag 102 may include a hole or tab 116 at the top to support the bag 102 from the hanger. Alternatively the bag 102 may include knobs on the side. The adjustable hanger may then include a rod with attachment connections, such a slip holes, to support the collection bag by the side knobs. Generally, any conventional means of supporting a urine bag 102 may be used with the adjustable hanger. The adjustable hanger permits the height of the collection bag 102 or output meter to be adjusted relative to the patient's bed. Various mechanisms may be used to adjust the height of the bag 102 or meter. The adjustable hanger may be used with many different types of beds or other structures, and is preferably used to reposition the height of the bag with respect to the height of the bed.

The adjustable hanger may be used to raise the collection bag and drain tube to minimize contact with the floor. An arm may connect the bed hook to the collection bag hook. The arm may include a hinge to permit the bag to swing upwards or downwards, to raise or lower the collection bag with respect to the floor. In an alternate embodiment, the arm may be vertical, and permit a user to pull the arm up to increase the height of the arm, expanding the distance from the meter to the floor. For example, the arm may be a sliding telescope. The arm may include engagement locations, such as holes or indentions, which a sleeve engages as the arm is extended. Another embodiment may permit the vertical arm to slide in a sleeve and have an engagement piece that supports the location of the arm relative to the sleeve. The engagement piece may be a clamp or may engage holes on the arm. Alternatively, the arm may be separate components that twist together. Therefore, the length may be determined by the amount the arm is screwed in or out. In one embodiment, a hinge may couple the bed hook with the arm of the adjustable hanger to permit position of the bag to move rotationally around a vertical axis.

Referring to FIG. 4A, the adjustable hanger 400 includes a first panel and second panel 418 connected by a hinge 434. The hook section 408 is attached to the first panel and the arm 410 with a “C” shaped coupling member is attached to the second panel. The adjustability of adjustable hanger 400 includes moving the first and second panels from a first position adjacent or in contact with one another, as shown in FIG. 4A, to a second position in generally parallel relation to one another, as shown in FIG. 4B.

Referring to FIG. 4C, the adjustable hanger 402 includes a telescoping member 424 that telescopes into a sleeve to adjust the distance separating the first hook section 408, attached to the sleeve, from the second hook section 412, attached to the telescoping member. In an alternate embodiment, the first hook section 408 is attached to the telescoping member and the second hook section is attached to the sleeve. It is noted that the first hook section 408 is facing in a direction opposite from the second hook section 412.

FIG. 4D shows another embodiment of an adjustable hanger, including a sliding member 420 with holes 426 to receive a pin positioned on a sleeve 430. The first hook section 408 is integral with the sleeve 430 and the second hook section 414 is attached to the sliding member 420 including holes 426 such that the distance between the first hook section 408 and second hook section 414 is accomplished by removing the pin from a hole in which it resides and sliding the sliding member 420 until another hole is aligned with the pin for insertion therein. The pin locks the position of the sleeve 430 relative to the sliding member 420. Of course, the first and second hook sections 408, 414 can alternatively be connected to the sliding member 420 and sleeve 430, respectively.

FIG. 4E illustrates another embodiment of an adjustable hanger, including a member 422 with a threaded section cooperating with threads on an internal surface of sleeve 432. Alternatively, the member 422 could have threads while the internal surface of the sleeve is threaded to receive the threads. The distance between the first hook section 408 and the second hook section 416 is adjusted by twisting the member 422 with respect to the sleeve 432. A locking member is envisioned in one embodiment to prevent relative movement between the member 422 and sleeve 432 once a suitable position has been established. The second hook section in this embodiment is coupled to the member 422 by a rotatable arm that receives a proximal portion of the member 422 or a portion extending therefrom. The rotatable arm enables correct positioning of the bag with respect to the structure from which the first hook section 408 is hanging following the twisting of the member 422 relative to the sleeve 432. The rotatable arm is selectively tightened and loosened with respect to the member 422 by a retaining tab. The retaining tab can operate in a threaded arrangement, for example, to tighten and loosen the rotatable arm.

The urine collection devices and systems described herein may also be included as part of a kit. The kit may include additional materials appropriate for using the devices. For example, a kit may include instructions for using the devices or systems. Instructions may be provided in any appropriate medium, including written, visual, pictographic, audible, or the like. In some versions, the instructions describe the methods of using the device as described above. Kits may also include additional materials (e.g., connectors, additional tubing, spare bags, etc.) useful in conjunction with the devices described herein. Sheets 1-4E show exemplary embodiments of a urine collection device described herein, including the features of a quick connect device, an outlet device, and an adjustable hanger.

While the design has been described in terms of particular variations and illustrative figures, those of skill in the art will recognize that the design is not limited to the variations or figures described. In addition, where methods and steps described above indicate certain events occurring in certain order, those of skill in the art will recognize that the ordering of certain steps may be modified and that such modifications are in accordance with the variations of the invention. Additionally, certain steps may be performed concurrently in a parallel process when possible, as well as performed sequentially as described above. Therefore, to the extent there are variations of the design, which are within the spirit of the disclosure, it is the intent that this description will cover those variations as well.

What is claimed is:

1. A fluid collection assembly, comprising:
a container having an interior chamber for collecting fluid; and
a quick connect device coupled to the top of the container to couple to a mated connection device on a drainage tube; wherein the quick connect device and the mated connection device are in an open configuration to permit fluid flow when engaged and are in a closed configuration to prevent fluid flow when disengaged.

2. The fluid collection assembly according to claim 1, wherein the container includes an outlet device coupled to the bottom of the container wherein the outlet device is in a normally closed configuration.

3. The fluid collection assembly according to claim 2, wherein the outlet device includes a valve biased in a closed position.

4. The fluid collection assembly according to claim 3, wherein the valve is selected from the group consisting of a trumpet valve, a duckbill valve, and a push button valve.

5. The fluid collection assembly according to claim 2, wherein the container includes a retaining device to hold an end of the outlet device

6. The fluid collection assembly according to claim 2, wherein the outlet device includes an interface to couple to a disposal container.

7. The fluid collection assembly according to claim 1, wherein the quick connect device comprises a male/female connection pair.

8. The fluid collection assembly according to claim 1, wherein the quick connect device comprises a duckbill and the mated connection device includes a female receiving portion that receives the duckbill to engage the quick connect device to permit fluid flow therebetween.

9. The fluid collection assembly according to claim 1, wherein the quick connect device further comprises a vented area.

10. The fluid collection assembly according to claim 1, further comprising an adjustable hanger attached to the container.

11. The fluid collection assembly according to claim 1, wherein the adjustable hanger includes an adjustable member to vertically reposition the container.

12. A fluid collection bag and drain assembly, comprising:
a container having an interior chamber for collecting fluid; and
an outlet device coupled to the bottom of the container wherein the outlet device is in a normally closed configuration and the container further comprises a retaining device to retain the outlet device.

13. The fluid collection bag and drain assembly according to claim 12, wherein the outlet device includes a valve to occlude the outlet device tube.

14. The fluid collection bag and drain assembly according to claim 12, wherein the outlet device includes an interface to couple to a disposal container.

15. An adjustable hanger attached to a fluid collection bag, comprising:
an adjustable member that vertically repositions the fluid collection bag;
a first hook section attached to the adjustable member to couple the hanger to a separate structure; and
a bag connection device attached to the adjustable member to couple the hanger to the fluid collection bag.

16. The adjustable hanger according to claim 15, wherein the adjustable member comprises a first panel connected to a second panel via a hinge, wherein the first hook section is connected to the first panel, and the bag connection device is connected to the second panel.

17. The adjustable hanger according to claim 15, wherein the adjustable member comprises a telescoping member telescoping into a sleeve, wherein the first hook section is connected to the sleeve, and the bag connection device is connected to the telescoping member.

18. The adjustable hanger according to claim 15, wherein the adjustable member comprises a sliding member positioned in a sleeve, the sliding member including spaced apart holes, the sleeve including a pin received in the holes to lock the sliding member relative to the sleeve.

19. The adjustable hanger according to claim 15, wherein the adjustable member includes a threaded member received in a sleeve having an inner surface including threads.

20. The adjustable hanger according to claim 19, wherein the bag connection device includes a rotatable arm.
